(19) United States
(12) Patent Application Publication
(10) Pub. No.: US 2005/0027223 A1
(43) Pub. Date: Feb. 3, 2005

(54) Digit splint for phalangeal fractures or mallet finger injury

(76) Inventor: Jimmy Phong Xuan Nguyen, Sydney (AU)

Correspondence Address: JACOBSON & JOHNSON, ONE WEST WATER STREET, SUITE 285, ST. PAUL, MN, 55107, US

(21) Appl. No.: 10/462,137

(22) Filed: Jun. 16, 2003

(51) Int. Cl. A61F 5/00

(52) U.S. Cl. 602/23

(57) ABSTRACT

There is a digit splint for treating injury to a finger 12. The splint includes a plurality of position adjustable segments 1A, 1B, 1C arranged end-to-end on a connecting linkage 2, 3 to form an elongated support securable along the dorsal or ventral surfaces of the finger. The segments are slidable on the linkage relative to one another in the longitudinal direction so that the splint can be adjusted to suit different finger lengths. The connecting linkage is deformable to put the finger in flexion for a phalangeal fracture or extension for a mallet finger injury.

### FIELD OF THE INVENTION

This invention relates to medical apparatus. More particularly although not exclusively it discloseas an improved form of digit splint for Phalangeal fractures or Mallet Finger injury.

### BACKGROUND TO THE INVENTION

Existing digit splints typically comprise a flat strip of malleable material such as aluminium which is cut to length, bent to a required shape depending upon the injury and then affixed along the dorsal surface of the finger using suitable padding and tape. Such splints however are difficult and laborious for the practitioner to cut and fit correctly. Moreover many patients find them uncomfortable due to the sharp edges reulting from the cutting and forming especially with Mallet Finger fractures. These fractures often require at least 8 weeks splinting of the distal phalangeal joint. A water resistant splint is also required which improves treatment compliance.

### SUMMARY OF THE INVENTION

It is therefore an object of this invention to ameliorate the aforementioned disadvantages and accordingly a digit splint for treating injury to a finger is disclosed, said splint including a plurality of position adjustable segments arranged end-to-end on a connecting linkage to form an elongated support securable along the dorsal or ventral surfaces of said finger, the segments being slidable on said linkage relative to each other in the longitudinal direction whereby the splint can be adjusted to suit different finger lengths and the connecting linkage being deformable to put said finger in flexion for a phalangeal fracture or extension for a mallet finger injury.

Preferably the segments are contoured to fit said dorsal surface.

It is further preferred that the segments are all of identical shape.

It is further preferred that the splint is securable along said dorsal surface using velcro straps placed around the finger.

It is further preferred that the connecting linkage comprise malleable metal pins or wires which are slidably received through apertures in said segments.

### BRIEF DESCRIPTION OF THE DRAWINGS

The currently preferred form of the invention will now be described with reference to the attached drawings in which:

FIGS. 1 and 2 are top and bottom perspective views of a digit splint according to this concept,

FIG. 3 is a detailed perspective view of a splint segment from one end (the other end being a mirror image), and

FIG. 4 is a cross-sectional view of the splint along the lines A-A of FIG. 1, and

FIGS. 5 and 6 are top and bottom perspective views of the splint when fitted to an injured finger.

### DETAILED DESCRIPTION OF THE PREFERRED EMBODIMENT

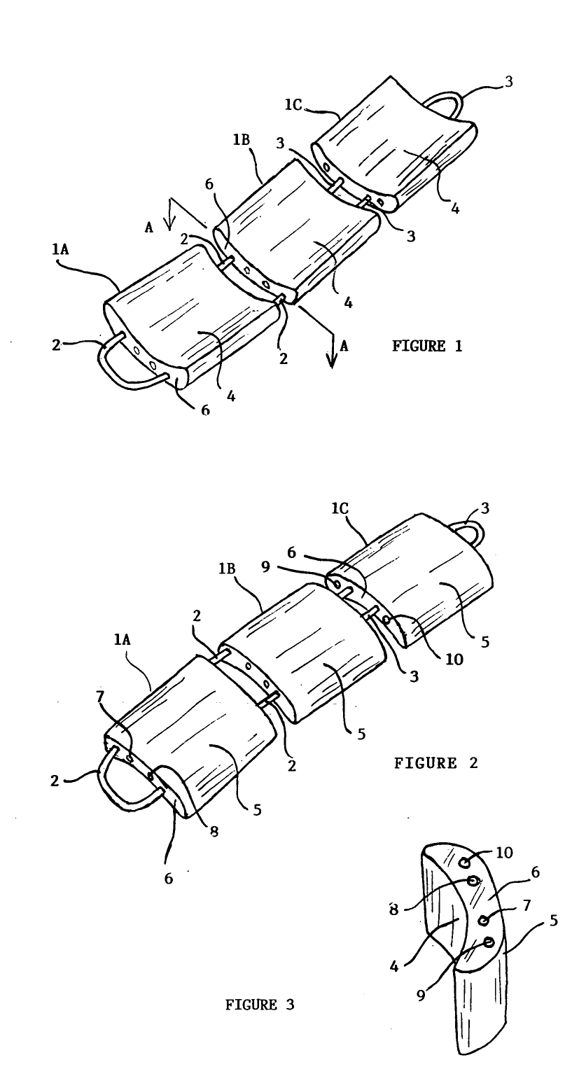

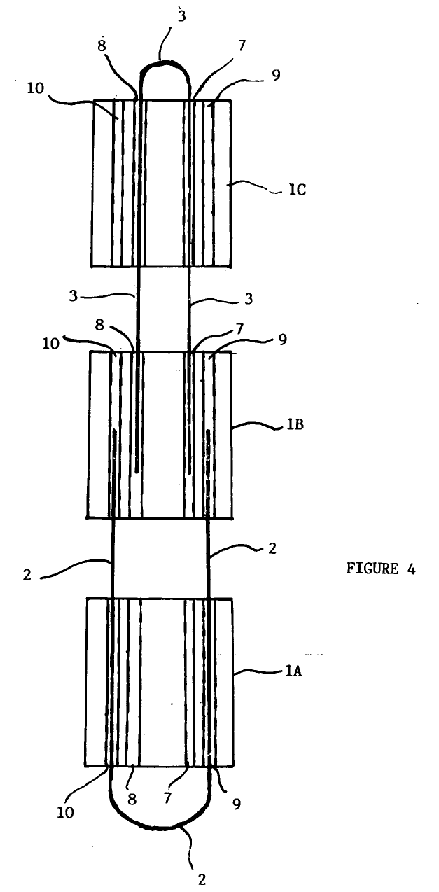

Referring first to FIGS. 1 to 4 the slint may comprise a set of three segments 1A, 1B and 1C which are arranged end-to-end on a connecting linkage formed by U shaped metal wires 2 and 3. With this embodiment the segments are preferably identical in shape. As best shown in FIG. 3 they each a have concave upper surface 4, rounded underside 5 and planar ends 6 through which two pairs of apertures 7, 8 and 9, 10 pass. These apertures are spaced symmetricaly about the longitudinal centre axis of the splint and extend completely through the length of the segments.

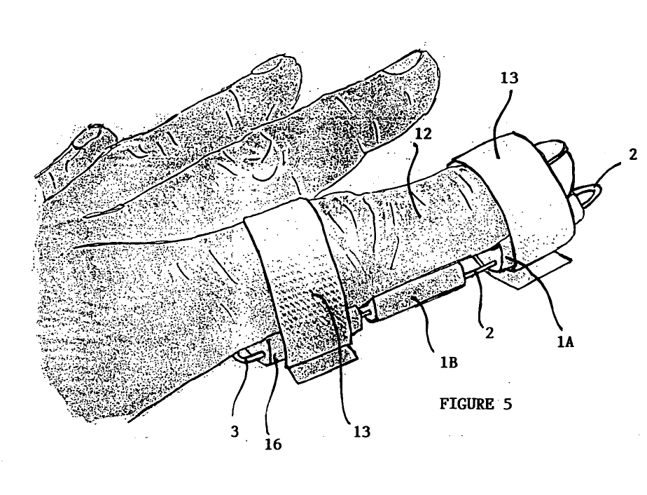

As shown in FIG. 4 the metal wires 2 and 3 are each shaped in the form of a U but are of different widths corresponding to the aforementioned pairs-of apertures. The narrow U shaped wire 3 passes from one end of the splint through the closely spaced apertures 7, 8 in segments 1C and 1B while the wider U shaped wire 2 passes from the opposite end through the other pair of apertures 9, 10 in segments 1C and 1B. The diameters of the wires and apertures are chosen to provide a close tolerance sliding fit so that an assembly is provided which while being longitudinally adjustable nevertheless resists lateral deflection. Each segment is slidable on the wires so that the assembly can be collapsed down from the expanded configurations shown to a shortened version where the ends of the segments closely abut each other. The length of the splint can thus be quickly and easily adjusted by the practitioner without the aid of tools.

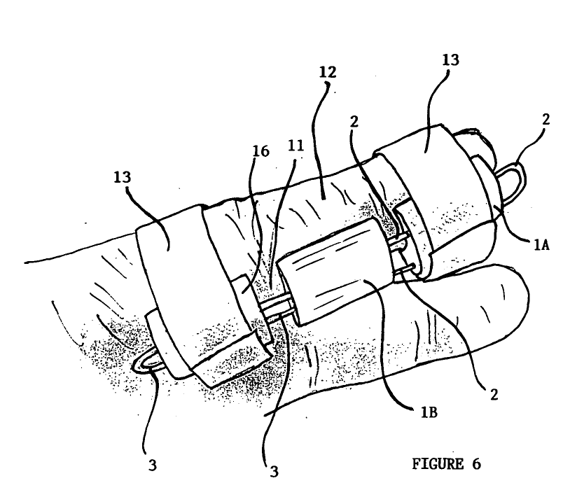

As shown in FIGS. 5 and 6 the splint, after adjustment for length, is fitted with the segment surfaces 4 engaging along the underside or dorsal surface 11 of an injured finger 12. Preferably the splint is held in place by velcro straps 13 or the like. As mentioned earlier the upper surfaces 4 of the segments are preferably concave so as to follow the contour of the dorsal surface and cradle the finger.

The finger 12 shown in FIGS. 5 and 6 is held straight as is required for some phalangeal fractures. The malleable nature of the mild steel connecting wires 2, 3 used with this embodiment however enables the splint to be bent so as to alternatively put the finger in flexion or in extension as required for Mallet Finger injuries. In the latter case the splint would also be modified from the example shown to use only two segments to hold the distal phalangeal joint in extension.

It will thus be appreciated that this invention at least in the form of the embodiment disclosed provides a novel and improved digit splint for finger injuries. Clearly however the example described is only the currently preferred form of the invention and a wide variety of modifications may be made which would be apparent to a person skilled in the art. For example the number, shape and the configuration of the segments and connecting wires may be changed according to design preference or following further development by the inventor. Also, while with the described embodiment the segments are moulded from plastic and the connecting wires are of mild steel the invention extends to the use of other suitable materials.

1. A digit splint for treating injury to a finger, said splint including a plurality of position adjustable segments arranged end-to-end on a connecting linkage to form an elongatable support securable along dorsal or ventral surfaces of said finger, the segments being slidable on said linkage relative to each other in the longitudinal direction whereby the splint can be adjusted to suit different finger lengths and the connecting linkage being deformable to put said finger in flexion for a phalangeal fracture or extension for a mallet finger injury.

2. The digit splint as claimed in claim 1 wherein in one position of use said segments have concave upper surfaces and planar ends through which apertures pass.

3. The digit splint as claimed in claim 2 wherein said connecting linkage is formed by metal wires which extend through said apertures in a close tolerance sliding fit.

4. The digit splint as claimed in claim 3 wherein said metal wires are of a malleable nature such that said splint can be bent by a practitioner without the aid of tools so as to put said finger in flexion or in extension.

5. The digit splint as claimed in claim 4 wherein the connecting linkage is formed by two metal wires which are formed into U shapes of different widths corresponding to respective pairs of said apertures.

6. The digit splint as claimed in claim 6 wherein said segments are all of identical shape.

8. The digit splint as claimed in claim 7 wherein there are two segments for holding a distal phalangeal joint in extension.

9. The digit splint as claimed in claim 8 wherein the splint is securable along said dorsal or ventral surfaces by the use of velcro straps placed around the finger.

10. The digit as claimed in claim 9 wherein said segments are moulded from plastic and the connecting linkage is of mild steel.
